(19) United States
(12) Patent Application Publication
(10) Pub. No.: US 2026/0151029 A1
(43) Pub. Date: Jun. 4, 2026

(54) Guiding A User To A Test Position For Performing A Computer-Based Eye Exam

(71) Applicant: EASEE HEALTH B.V., Amsterdam (NL)

(72) Inventors: Jouke-Thiemo WALESON, Amsterdam (NL); Fehim ISMAILOV, Amsterdam (NL)

(21) Appl. No.: 18/705,676

(22) PCT Filed: Nov. 1, 2022
(86) PCT No.: PCT/NL2022/050614

§ 371 Date: Apr. 29, 2024

(30) Foreign Application Priority Data
Nov. 1, 2021 (NL) 2029579

(51) Int. Cl. A61B 3/00; A61B 3/02

(52) U.S. Cl. CPC A61B 3/0083; A61B 3/02

(57) ABSTRACT

A computer-implemented method for guiding a user to a test position, including displaying a symbol on the screen that is spaced apart from a reference item; varying the distance between the symbol and the user, while having one eye closed and looking at the reference item, until an initial position is reached wherein the symbol is invisible; receiving an input indicating that the user is at said initial position; correlating a parameter or object, as recorded by the camera while the user is at the initial position, to the distance; based on the correlation, determining the unknown camera parameter; and instructing the user to move to the test position for performing an eye test, while determining a current distance between the user and the screen based on a change in the camera recordings.

### BACKGROUND

The present invention relates to a computer-implemented method for guiding a user to a test position at a test distance from a computer screen, while using a camera having at least one unknown camera parameter.

Online eye exams, performed on a computer of a user without the help of a professional assistant and preferably without the help of any assistant, have been described in the patent literature since about the year 2000 and are more and more accepted by consumers. One of the first patent applications relating to this particular field is EP1296588A1. Other patent applications related to this particular field are EP2967316A1 and EP3655314A1, the latter being in the name of the same applicant as the present patent application. The methods described in these patent applications all suffer from the same problem: when it is not known in advance on which type of computer the test is performed, it is very difficult to determine the distance between the user and the screen on which the test is displayed with a reasonable amount of certainty. This results from the fact that many types of computers are available, each having a (semi-)unique set of characteristics. The less accurate the test distance can be determined while performing the eye exam, the less reliable the prescription resulting from the exam. Therefore, it is of crucial importance to reliably determine a distance between the user and the screen.

In EP1296588A1 the user is asked to measure the distance between the screen and the test position him/herself. However, no way of checking whether the user is actually at the correct position is provided for in this patent application, so if the user is “cheating” and is not at the prescribed distance, an incorrect prescription will result. This effectively would render the test useless.

In EP2967316A1 the camera is calibrated by holding a credit card at 11 inches from the screen. The user has to hold up the credit card and determine the 11 inches him/herself. This process of measuring the distance between the screen and the card, holding up the card, and simultaneously looking at the screen all at the same time is rather inconvenient for the user, and additionally has a high margin of error.

In EP3655314A1 the camera is calibrated using statistical information about the average pupillary distance of users and the average initial distance at which a computer screen is naturally viewed when starting up the test. In this way, the user-screen distance can be determined with about 8-10 % accuracy.

A need however remains to determine the distance between the screen and the user with more precision, reliability and/or with a more seamless user experience.

### SUMMARY OF THE INVENTION

Accordingly, the present disclosure relates to a computer-implemented method for guiding a user to a test position at a test distance from a computer screen, wherein use is made of a camera associated with the computer screen, the camera having at least one unknown camera parameter, the method comprising the steps of:
displaying, on the computer screen, at least one symbol that is spaced apart from a reference item by a symbol distance;
varying a distance between the user and the symbol, while instructing the user to close and/or shield one eye and look at the reference item with the open eye, until an initial position is reached wherein the displayed symbol is not visible to the user, said initial position defining a first user-screen distance depending on the effective symbol distance;
receiving an input from the user, the input indicating that the user is at said initial position;
while the user is at the initial position, recording one or more parameters or objects with the camera, and correlating said recordings to the first user-screen distance;
based on said correlation, determining at least one unknown camera parameter; and
instructing the user to move from the initial position to the test position, for performing an eye test at said test position, while determining a current user-screen distance based on the determined camera parameter and a change in the camera recordings.

Advantageously, in the presently disclosed method use is made of the blind spot of a user to determine the initial position between the user and the screen. It is found by the present inventors that the angle between the centre of vision and the blind spot area of a human eye is highly constant among all individuals of the human population, at about 13.6 degrees (corresponding to the “start” or “edge”of the blind spot region) to 15.0 degrees (corresponding to the centre of the blind spot region). Thus, while carrying out the method as presented herein, when the user indicates that he or she is in a position wherein the displayed symbol is invisible, and when the distance between the reference item and the displayed symbol is known, the distance between the user's eye and the screen can be calculated with great precision.

At this position, referred to herein as the “initial position”, e.g. the pupillary distance of the user as visible on an image obtained by the camera may be determined, based on which determination e.g. the focal length of the camera can be obtained as the pupillary distance is also relatively constant among the human population and as the relations between object distance, the focal length, the image distance, size of the object and size of the image are well-established.

As an alternative, at the initial position the user may be asked to place a card of known size, e.g. a credit card (sized card), on his/her forehead or before his/her eyes. Again, based on the size of the card on the camera recording and the physical size of the card, using standard optical formulas, the focal length of the camera may be obtained with great precision.

In this way, it is possible to obtain the focal length of the camera. Knowing said focal length, the distance of the user to the computer screen can be determined with a high accuracy at any time throughout the eye test/exam, i.e. also when the user moves away from the initial position.

It is noted that the thus-obtained focal length of the camera might be rounded to correspond to known focal lengths of camera's. The inventors have realized that although many different camera systems for computers exist, only a relatively select number of focal lengths are found in this group of camera systems. If for example a focal length is determined to be 49.2 mm with the method as described herein, but no such camera's are known, the focal length may be set at 50.0 mm as camera's with such a focal length are known.

Also the field of view of the camera is an important parameter, as this is a measure for the degree with which a user-specific parameter changes when the user moves with respect to the screen. The field of view can be determined directly or indirectly once the focal length of the camera is known. The focal length of a lens defines the lens's angular field of view. For a given sensor size, the shorter the focal length, the wider the angular field of the lens (and vice versa). To mention one extreme example of the effect of the field of view of a lens: a fisheye camera lens will have quite some distortion, especially near the edges of the image received by it. Preferably, for such lenses, but also for other lenses, it is prevented that a user is in the edge of the image viewed by the lens to minimize distortion.

Further advantageously, with the present method the distance between the user and the screen may be determined with the use of only a single camera.

In accordance with the presently disclosed method, after the initial distance between the user and the computer screen has been determined with the “blind spot test”, the user is guided away from that initial distance to a test position at a test distance. For example, the test distance can be in between 200 and 450 cm, to perform a “far vision” test. In other examples, the test distance can be in between 40 cm and 70 cm, to perform a “computer vision test”. In yet other examples, the test distance can be in between 20 cm and 40 cm, to perform a “near vision” test. Of course, during the eye exam, all three tests can be performed, e.g. sequentially. Alternatively, one or two of these tests may be performed.

When guiding the user from the initial position to the test position, the current distance between the user and the screen can be determined with high precision as the (initially unknown) camera parameters are known from the calibration/correlation performed while the user was positioned at the initial position. Also, user-specific parameters may be correlated with the image as viewed by the camera at the initial position, such that also these user-specific parameters may be used to allow future determination of a current distance between a user and a screen on which the test is performed. More in particular the change in these user-specific parameters in the image viewed by the camera as the user moves from the initial position is relevant.

The distance at which the eye test is performed may in embodiments be fixed (within a few centi-or decimeters), the user being guided to the test position step by step.

In other embodiments the test distance at which the eye test is performed is relatively variable, the user e.g. being asked to “set an X number of steps backwards” from the initial position, and the exam as displayed on the screen being scaled up or down depending on the (absolute) size of the steps taken by the user. This is made possible as the current distance between the user and the screen can always be determined with great precision. The number X may e.g. be a variable in between 2 and 20, and may depend on the size of the room in which the user performs the test.

While performing the method, use is made of a camera that is associated with the computer screen. For example, the camera can be built-in into the computer screen, or the camera may e.g. be a webcam that is linked to the computer screen and/or integrated with the computer screen. It may be assumed that the distance between the screen and the camera is small. If this distance is substantial, this latter distance should be accounted for to obtain the user-screen distance from the camera recordings. In this latter case the user may e.g. indicate the distance between the camera and the screen manually.

While performing the method, use is made of a computer screen. For example, the computer screen can be a screen of a laptop, an external screen linked with a laptop, a screen of a smartphone, a screen of a tablet, or any other computer screen. As stated in the background section, when offering an eye exam as an online test, to be performed on a computer screen of the user, it is not known in advance which screen the user has, nor is it known which camera the user has. As properties of screens and cameras vary widely, some calibration has to be performed to allow the eye exam to be performed as accurate as possible. One known method to obtain screen properties is to ask the user to place a credit card—or another card of the same size—on the screen and manipulate a rectangle until the card precisely fits into the rectangle. As a credit card has the same size everywhere in the world, the number of pixels per unit length (cm, inch) can be determined from this test by a computer program.

To calibrate the camera, the blind spot calibration method as disclosed herein may be used. From the blind spot calibration method, as explained in the above, e.g. the focal length and/or the field of view of the camera—which are unknown before the calibration method—can be obtained. Knowing the focal length and/or the field of view of the camera as well as the physical value of a user-specific paramater, the distance between the user and the screen can easily be calculated at any time, using standard optometric formulas and the image recorded by the camera.

According to the method as disclosed herein, at least one symbol is displayed on the computer screen. The symbol is spaced apart from a reference item. For example, the reference item may be another symbol displayed on the screen, such that in total at least two symbols are displayed. Alternatively, the reference item may e.g. be the edge of the computer screen/the monitor.

According to one embodiment the method, the distance between the symbol and the user is varied by instructing the user to move with respect to the symbol. For example, the user can move backwards and/or forwards with respect to the computer screen, increasing resp. decreasing the distance between the user and the screen, while e.g. maintaining a fixed symbol distance between the reference item and the symbol, until the user is at a position/distance wherein the symbol is not visible with one eye closed.

According to an alternative embodiment of the method, the distance between the symbol and the user is varied by instructing the user to remain fixed at his/her position in front of the screen, while moving the symbol to the left and/or to the right until the symbol is not visible with one eye closed.

According to yet another embodiment of the method, the distance between the symbol and the user is varied by automatically moving the symbol to the left and the right on the screen, e.g. while instructing the user to remain at a fixed position.

Of course, a combination of these alternative embodiments may also be used, e.g. the user and the symbol both moving, automatically or manually.

When the symbol disappears from the eye field of the user, the user indicates this by providing an input. From the symbol distance at that time, combined with the knowledge that the angle between the centre of vision and the blind spot area of a human eye is highly constant among all individuals of the human population and is always about 13.6-15.0 degrees (start of the blind spot region resp. centre of blind spot region), the momentaneous distance between the user and the screen can be obtained easily and accurately. With that user-screen distance accurately known, as explained in the above, the focal length and/or field of view of the camera can be obtained by e.g. relating the present pupillary distance as projected on the image viewed by the camera, to the actual pupillary distance of the user, said actual pupillary distance again known to be highly constant among all individuals of the human population.

When calibrating the unknown camera parameter by correlating the user-screen distance at the initial position to one or more parameters or objects recorded by the camera, it is recommended to use a parameter or object of which the physical value is as constant as possible, amongst all users. Examples include a credit card or other card with the same dimensions owned by the user and the distance between pupils of the user.

On the other hand, once the calibration of the camera parameter is performed and the focal length and/or field of view of the camera is known, the physical value of other user-specific parameters may optionally be obtained while the user is at the initial position. Such additional parameters, which may significantly vary between individual users, may be used during the remained of the method to track the momentaneous user-screen distance. Preferably, any such parameters are non-variable while performing the test. For example, the should-to-shoulder width may be logged at the initial distance, or the ear-to-ear distance, the eyebrow-to-eyebrow distance, the mouth width, the eye white-white distance, arm length, and other similar parameters. Knowing the image size of these parameters and the focal length/field of view of the camera, the “true” size of these parameters may be obtained with the user in the initial position. When these true sizes are logged, they can be used to determine the distance between the user and the screen when the user moves away from the initial position. Advantageously, if at any point the user moves to a position wherein the pupillary distance can no longer accurately be determined from the camera footage, or when the user closes one eye to perform a test with the opened eye, any such parameters may be used as a further indication for calculating the current distance between the user and the screen.

Hence, it is not necessary that the same parameter/object is used in the “correlation” step and the “tracking” step.

At the test position, an eye test is provided to the user. Such eye tests or eye exams have been described at length in the prior art and may e.g. include the determination of the refractive error, the angle of astigmatism, and any other aspect of an eye prescription.

In embodiments, to more accurately determine the user-screen distance at the initial position, the “blind spot test” as described in the above may be performed for both the left and the right eye before the unknown camera parameter is determined. If the results of the two tests are too far apart another test may be performed. If they are within a pre-defined error margin e.g. an average value may be taken.

In embodiments, the step of varying the distance between the user and the displayed symbol is preceded by a step of asking whether the user is currently wearing glasses or contact lenses and, if so, what the prescription of said glasses/lenses is. It is found by the present inventors that glasses and/or contact lenses break the incoming light rays before they are received by the human eye. As a result of this light ray breaking, the angle between the centre of vision and the blind spot area of a human eye may be different from the above-mentioned 13.6 degrees; the precise difference depending on the strength of the glasses/contacts. Therefore, preferably any such difference is accounted for in the calculation of the initial distance by asking the user to indicate whether he or she is wearing his or her glasses/contacts while performing the test and, if so, what the current prescription is.

In embodiments, the at least one unknown camera parameter includes the focal length and/or the field of view of the camera. The relevance of the focal length and the field of view has already been explained in the above.

In embodiments two symbols are displayed on the computer screen in the displaying step to determine the initial position and the first user-screen distance, each of the displayed symbols preferably having a different shape and one of the symbols being the reference item. This helps the user to distinguish between the reference item, the symbol at which the user should look with the open eye, and the other symbol, that is to disappear. For example, one of the two symbols may have the shape of a “plus” or a “cross”, whereas the other of the two symbols may have the shape of a “circle”. Also, the symbols may have mutually different colours.

In embodiments, the cross sectional size of the symbols may be less than 2.5 cm, e.g. about 1 cm. This on the one hand ensures that the symbols are well visible from a moderate distance from the screen, while it on the other hand ensures that the symbols actually disappear when one eye is closed and the symbol is in the blind spot region (the latter would not be the case when the symbols would be too large).

In embodiments, the symbol distance initially is in the range of 5 cm-50 cm. As stated, following the initial display of the symbol(s), the user may move backwards and/or forwards in front of the screen and/or the symbol distance may be altered by either manually moving the symbol to the left and/or to the right or by having a computer program moving the symbol to the left and/or to the right such that the symbol distance may vary while carrying out the disclosed method.

In embodiments the initial position, the position at which the user stands when the reference symbol disappears, may be located at a user-screen distance of in between 20 cm and 200 cm. As stated, once the calibration of the camera parameter(s) is performed, the user is typically asked to move away from the initial position, to a test position at a test distance, to perform the eye exam. In rare cases it might occur that the desired test position is exactly at the initial position, so that the instruction simply states something along the lines of “please remain at position to perform the first phase of the eye exam”.

In embodiments, the input provided by the user is provided via a smartphone of the user, the smartphone being held by the user at the initial position and the smartphone being linked with the computer program displaying the at least one symbol on the screen. This has the advantage that the user can remain at the test position while providing the input and does not have to move back and forth with respect to the screen all the time.

In embodiments, the focal length and/or the field of view of the camera are determined in the determining step. As stated in the above, these camera parameters are typically unknown for the camera that a randomly-selected person is using when performing an online eye exam on his or her computer, as a great many different number of computers and associated cameras have been circulated worldwide and, on top of that, the number increases on a daily basis. It is found by the present inventors that only a relatively limited number of camera focal lengths are used by the different available cameras, said focal lengths having discrete intervals. So, based on the correlation step the focal length of the camera may be estimated, upon which it may be possible to look up which focal lengths are known for cameras in circulation and the closest matching number can be selected to make the distance calculation even more precise. Of course, any such look-up table must be updated regularly. As described in the above, once the focal length is known, the field of view of the camera can be obtained as well.

In embodiments, the instructions to guide the user to the test position are provided by displaying instructions like “move away from the computer screen” and/or “move closer to the computer screen” on a screen of a smartphone and/or on the computer screen that displayed the symbol.

In alternative embodiments, the instructions to guide the user to the test position are provided by vocal instructions, played by a speaker of the smartphone and/or by a speaker of the computer.

In embodiments, the method is performed by a computer program executed by a computer.

A second aspect of the present invention relates to a computer program or computer program product comprising computer program code which, when the program code is executed by at least one processor of a computer, causes the computer to perform the steps of the method as described in the above.

These and other aspects of the present disclosure will now be elucidated further with reference to the attached figures. In said figures, same or like elements will be indicated with the same reference numbers.

### BRIEF DESCRIPTION OF THE FIGURES

FIG. 1 schematically shows an optional method step of calibrating a computer screen;

FIG. 2 schematically shows one possible embodiment of a method step of displaying at least one symbol on the screen;

FIG. 3 schematically shows a user viewing the symbol displayed on the screen;

FIG. 4 schematically shows one possible embodiment of a method step of the user moving relative to the symbol;

FIG. 5 schematically shows one possible embodiment of a camera determining a distance between the user and the screen while the user is at a position wherein a reference item is invisible;

FIG. 6 schematically shows different test positions for a user, each position corresponding to a different distance from the screen; and

FIG. 7 schematically illustrates displaying an eye test on the screen.

### DETAILED DESCRIPTION OF THE FIGURES

The present invention relates to performing an eye test on a computer screen of a user, preferably without any help of others. A problem when offering such a test, e.g. when selling the tests in an online context, is that it is not known in advance which type of screen the user has, and additionally it is a problem to accurately determine how far the user is from the screen when performing the test. The latter problem is in part due on the fact that besides the screen characteristics also the camera characteristics of the user's camera are not known.

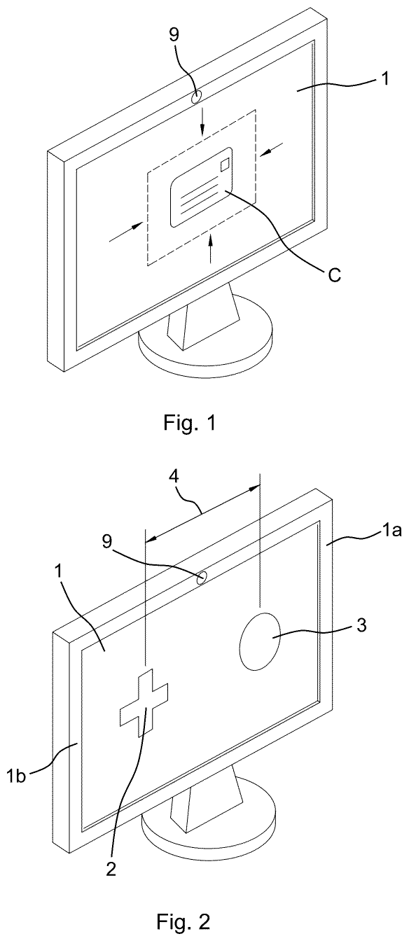

Presently available tests typically start with a step to calibrate the screen size, by determining the amount of pixels per length unit (e.g. cm or inch). To do this, a user is asked to put a card C of a known size, such as a credit card, on his or her computer screen 1 and to manipulate a rectangle until the rectangle fits around the card. Such a calibration step is shown in FIG. 1, wherein it is noted that this screen calibration step may be performed in many other ways and is optional in the context of the presently disclosed method. Shown as well in FIG. 1 is a camera 9, the camera 9 needing calibration of some sort as well as not all parameters of the camera 9 are known. In the shown embodiment, the camera 9 is integrated with the computer screen 1, as is more and more common on e.g. iMac's and laptops. In other embodiments, the camera 9 may be an external camera, e.g. a webcam, that is connected to the screen 1/monitor.

In an optional and non-shown step, e.g. following the screen calibration step as described in the above but possibly preceding said screen calibration step, the user may be asked whether he or she is currently wearing glasses and/or contact lenses. If the answer to said question is in the affirmative, the next question will be the corrective strength of the glasses/contact lenses.

In a next step, to perform said camera calibration as accurately as possible, a symbol 3 is displayed on the computer screen 1. Besides the symbol 3, there is also a reference item 1a, 1b, 2. In embodiments, the reference item may be the edge 1a, 1b of the monitor. In such embodiments, the reference item 1a, 1b is not displayed on the screen 1 but is e.g. associated with the screen 1. In the shown embodiment the reference item is displayed as a second symbol 2 on the screen 1. In the present embodiment, the reference item 2 and the symbol 3 have a different shape, but this is not required per se. It is however convenient, as this helps the user in not mixing up the symbol 3 at which he or she should look when the camera calibration is performed (to be explained in more detail below). The symbol 3 and the reference item 2 are spaced apart from each other by a symbol distance 4. This symbol distance 4 may initially e.g. be between 5 and 50 cm, wherein the maximum distance obviously mainly depends on the physical dimensions of the screen 1 available to the user (not known in advance when the method is sold to a user) and wherein the minimum distance shouldn't be too low as then the user-screen distance may not be accurately determined as a result of an inaccurate blind spot test.

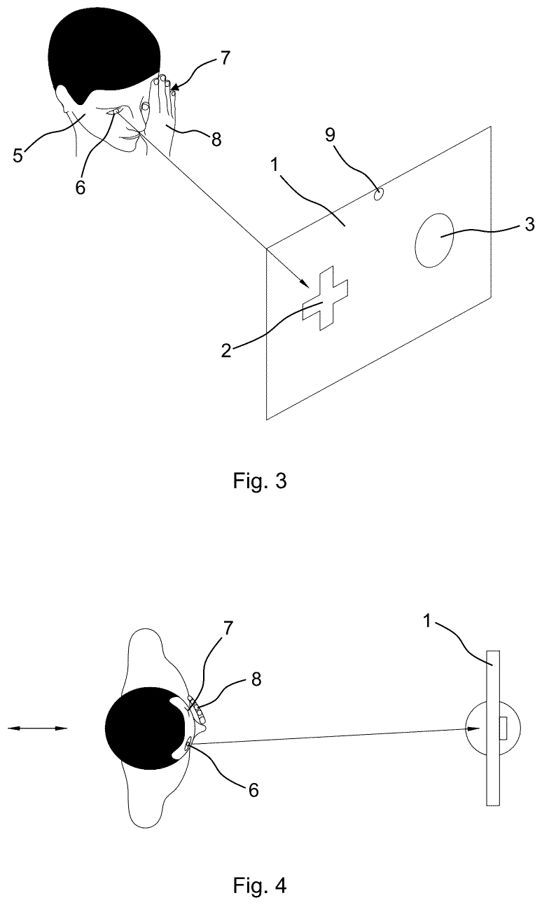

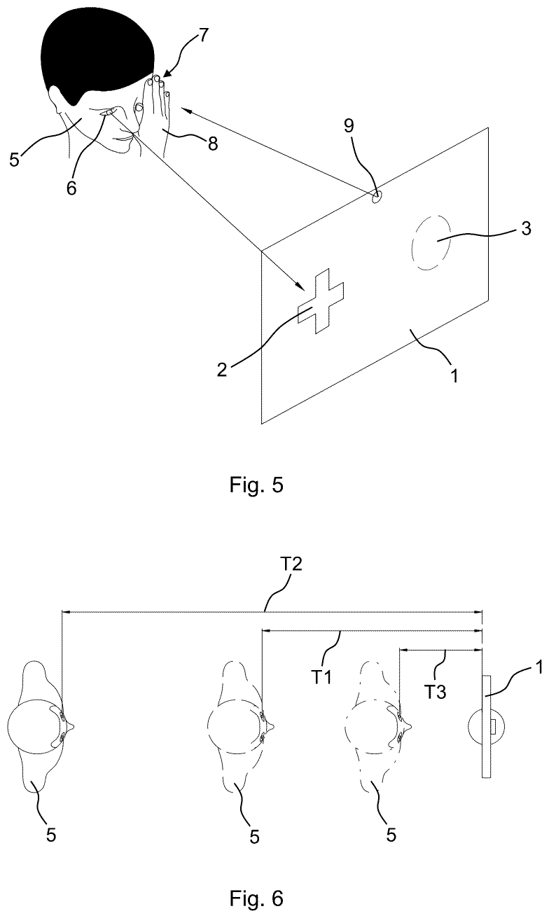

Turning now to FIG. 3, a user 5 is shown with one open eye 6 and one closed eye 7, the closed eye 7 here being shielded by a hand 8 of the user 5. With the open eye 6, the user 5 looks at the reference item 2. With reference to FIG. 4, the user 5 may move backwards and/or forwards in front of the screen 1 while keeping a fixation on the reference item 2 with the opened eye 6, increasing and decreasing his or her distance from the screen 1. This user 5 continues to move until the displayed symbol 3 is no longer visible to the user 5. It is noted that in reality, if the screen 1 would be looked at from another distance or if the closed and covered eye 7 would be opened and uncovered, the symbol 3 would be visible but at a specific distance with one eye covered, because of the blind spot of the opened eye 6, a symbol 3 can disappear from sight. This situation is shown in FIG. 5, with the displayed symbol 3 is shown in dotted lines to indicate its disappearance (to the user 5) thereof.

It is noted that, besides the user 5 moving in front of the screen 1, also the symbol distance 4 may be varied to place the displayed symbol 3 in the blind spot of the user 5. For example the symbol 3 may be moved to the left and/or to the right on the screen while the user 5 continues to look at the reference item 2 with the opened eye 6.

As another example, the symbol 3 may be moved to the left and to the right by a computer program running on the background.

The distance between the user 5 and the screen 1 at which the user 5 no longer sees the displayed symbol 3 will hereafter be referred to as the “initial position”. This initial position depends (solely) on the symbol distance 4 at that time. At the initial position, both the symbol distance and the user-screen distance are accurately known. The present inventors have found that the angle between the centre of vision and the blind spot area of a human eye is highly constant among all individuals of the human population, at about 13.6 degrees (when considering the “start”/“edge” of the blind spot region)/15.0 degrees (when considering the centre of the blind spot region). Thus, with the user in the position wherein the blind spot effect is noticeable (i.e. the symbol 3 disappearing from sight), with this angle known and constant among the human population as well as the current symbol distance known, the distance between the user 5 and the screen 1 can be determined with high accuracy. The program executing the presented method knows that the user 4 is at the initial position as this is indicated on an input device by the user, the input device e.g. being a smartphone of the user 5 that is coupled with the software that is used to display the symbols 2, 3 on the screen 1.

For example, the user-screen distance at the initial position may be between 20-200 cm from the screen 1.

With the distance between the user 5 and the screen 1 exactly known, the camera 9 may be calibrated. For example, knowing that the distance between the centres of the pupils of humans is a relatively constant absolute value across the entire human population, at about 65-67 mm, and when determining the size of that same parameter on the image as captured by the camera 9, the focal length of the camera 9 can be determined using standard optometric formula's. From the focal length, also the field of view of the camera can be obtained.

As an alternative to using the semi-fixed parameter of pupillary distance, the user may be instructed to hold up an object of known dimensions, e.g. a credit card or a card with the same dimensions near or at the eyes of the user. This also allows to obtain the focal length of the camera based on the user-screen distance, the recordings of the camera, and the known physical dimensions of the object.

Knowing the focal length, the field of view and the user-screen distance at the initial position, also the size of other parameters for the user 5 may be obtained, now from their size on the recordings of the camera 9. Preferably any such parameters are constant for the user, for the duration of the subsequently performed eye exam, but the parameters may be non-constant when the entire human population is concerned. For example, once the focal length and the field of view of the camera 9 are known the shoulder-to-shoulder width of the user may be determined; the eyebrow-to-eyebrow width, the eye white-to-eye white width, the ear-to-ear width, the mouth width, and many other parameters. Preferably, any such parameters remain constant during the eye exam to be performed; e.g. any parameter related to the haircut may be less suitable as these parameters may changes when the user fiddles with his or her hair during the course of the eye exam. Advantageously, such parameters that are determined in second instance may be used to calculate a current user-screen distance when the user 5 has moved away from the initial position and/or when the pupillary distance cannot be determined from the recordings of the camera 9, e.g. as one eye is closed or because the user is too far from the screen 1 to reliably determine the number of pixels between the pupil centres.

In other words, the parameter/object used in the “correlation” step for the purpose of calibrating the unknown parameter may differ from the parameter used in the “tracking” step for the purpose of determining a user-screen distance as the user moves away from the initial position.

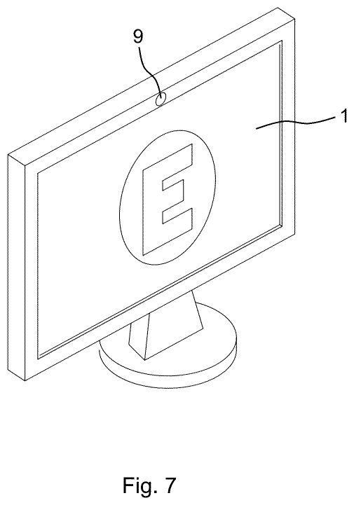

Turning now to FIG. 6, after having calibrated the camera 9 and, optionally, having determined some parameters of the user 5, said user 5 is instructed to move from the initial position T1 to a test position T2, T3. Typically, the test position T2, T3 is at a different location than the initial position T1. There are (at least) two ways to guide the user 5 to the test position T2, T3.

Firstly, it is possible to instruct the user 5 to take a number of steps from the initial position, e.g. five steps backwards (i.e. away from the screen 1), or e.g. one or two steps forwards (i.e. to the screen 1). The step size of the user 5 is not a prior known, but as the distance between the user 5 and the screen 1 can be determined at all times based on the change of the user parameters in the recordings of the camera 9 once the relevant camera parameters are known, the test as displayed on the screen 1 can be modified (scaled up or scaled down) depending on the exact distance between the user 5 and the screen 1 at the time of performing the eye exam.

Secondly, it is possible to guide the user 5 to a pre-determined location, at a pre-determined distance from the screen 1, for carrying out the eye test(s). This may be done by continuously instructing the user 5 to move relative to the screen 1 until a desired test location T2, T3 is reached.

This guiding/instructing can, again, be done in a number of ways. For example, voice commands may be provided through a microphone of the computer. For example, voice commands may be provided through a microphone of a mobile device of the user 5 (e.g. the input device). For example, text may be displayed on the computer screen 1 on which the eye test is to be performed. For example, text may be displayed on a mobile device of the user 1.

For example, the eye test may comprise a “far vision” test, typically performed at 150 cm- 450 cm from the screen 1.

For example, the eye test may comprise a “computer vision” test, typically performed at 40-70 cm from the screen 1.

For example, the eye test may comprise a “near vision” test, typically performed at 20-40 cm from the screen 1.

Performing the eye exam is schematically illustrated in FIG. 7. It should come as no surprise to one skilled in the art that any kind of eye test may be performed and that the invention is in no way limited to what type of test is performed once the user 5 is at the test position T2, T3.

In this way, a user 5 can be guided to a test position T2, T3 for performing an eye test, the test position T2, T3 at a test distance from a computer screen 1. When using the method as explained herein, it is not needed to know all parameters of the camera 9 that is used to determine the distance in advance.

### LIST OF REFERENCE NUMERALS

1 Screen
1a monitor edge
1b monitor edge
2 reference symbol
3 displayed symbol
4 symbol distance
5 user
6 open eye
7 shielded eye
8 hand
9 camera
C credit card
T1 initial position
T2 test position
T3 test position

1. A computer-implemented method for guiding a user (5) to a test position (T2, T3) at a test distance from a computer screen (1), wherein use is made of a camera (9) associated with the computer screen (1), the camera (9) having at least one unknown camera parameter, the method comprising the steps of:
displaying, on the computer screen (1), at least one symbol (2, 3) that is spaced apart from a reference item (1a, 1b, 2) by a symbol distance (4);
varying a distance between the user (5) and the symbol (2, 3), while instructing the user to close and/or shield one eye (7) and look at the reference item (1a, 1b, 2) with the open eye (6), until an initial position (T1) is reached wherein the displayed symbol (3) is not visible to the user, said initial position (T1) defining a first user-screen distance depending on the effective symbol distance (4);
receiving an input from the user (5), the input indicating that the user (5) is at said initial position (T1);
while the user (5) is at the initial position (T1), recording one or more parameters or objects with the camera, and correlating said recordings to the first user-screen distance;
based on said correlation, determining at least one unknown camera parameter; and
instructing the user (5) to move from the initial position (T1) to the test position (T2, T3), for performing an eye test at said test position (T2, T3), while determining a current user-screen distance based on the determined camera parameter and a change in the camera recordings.

2. The method according to claim 1, wherein the step varying a distance between the user (5) and the symbol (2, 3) is preceded by a step of asking whether the user (5) is currently wearing glasses or contact lenses.

3. The method according to claim 1, wherein the at least one unknown camera parameter includes the focal length and/or the field of view of the camera (9).

4. The method according to claim 1, wherein the camera (9) is a camera (9) that is built into the computer screen (1), a webcam integrated with a computer comprising the computer screen (1) or a webcam mounted to the computer screen (1).

5. The method according to claim 1, wherein two symbols (2, 3) are displayed in the displaying step, each of the displayed symbols (2, 3) having a different shape and one (2) of the two displayed symbols (2, 3) being the reference item.

6. The method according to claim 1, wherein the symbol distance (4) initially is in the range of 5 cm- 50 cm.

7. The method according to claim 1, wherein the initial position (T1) is located at a distance of in between 20 cm- 200 cm from the screen (1).

8. The method according to claim 1, wherein the input provided by the user (5) is provided via a smartphone of the user (5), the smartphone being held by the user (5) at the initial position (T1) and the smartphone being linked with a computer program displaying the at least one symbol (2, 3) on the screen (1).

9. The method according to claim 1, wherein in the recording step a user-related object is used, e.g. a card of a known size that is held to the forehead of the user (5) or a card of a known size that is held in front of the eyes of the user (5).

10. The method according to claim 1, wherein in the recording step a user-specific parameter is recorded, said user-specific parameter including at least one parameter from the group comprising the parameters pupillary distance, eye white to white distance, shoulder to shoulder distance, ear to ear distance, eyebrow to eyebrow distance.

11. The method according to claim 1, wherein the instructions are provided by displaying instructions like “move further away from computer screen” and/or “move closer to computer screen” on a screen of a smartphone linked with the computer and/or on the computer screen (1) that displayed the symbols (2, 3).

12. The method according to claim 1, wherein the instructions are provided by vocal instructions, played by a speaker of the smartphone and/or by a speaker of the computer.

13. The method according to claim 1, wherein the test position (T2) is at a distance of in between 150 cm- 450 cm from the screen (1).

14. The method according to claim 1, wherein the test position (T3) is at a distance of in between 20 cm- 40 cm from the screen (1).

15. The method according to claim 1, wherein the test position is at a distance of in between 40 cm- 70 cm from the screen (1).

16. The method according to claim 1, wherein the method is performed by a computer program executed by a computer.

17. A computer program or computer program product comprising computer program code, which when the program code is executed by at least one processor of a computer, causes the computer to perform the steps of a method according to claim 1.
